(19) United States
(12) Patent Application Publication
(10) Pub. No.: US 2009/0317796 A1
(43) Pub. Date: Dec. 24, 2009

(54) Indirect immunofluorescence assay typing kit for coxsackievirus A group and method for typing coxsackievirus A group

(75) Inventors: Tsuey-Li Lin, Taipei City (TW); Tsan-Chang Tseng, Taipei City (TW)

Correspondence Address: Muncy, Geissler, Olds & Lowe, PLLC, P.O. BOX 1364, FAIRFAX, VA, 22038-1364, US

(73) Assignee: Centers for Disease Control Department of Health

(21) Appl. No.: 12/213,540

(22) Filed: Jun. 20, 2008

(51) Int. Cl. C12Q 1/70

(52) U.S. Cl. 435/5

(57) ABSTRACT

An indirect immunofluorescence assay typing kit for coxsackievirus, comprising: a first reagent comprising a mixture of an anti-coxsackievirus A2 polyclonal antibody, an anti-coxsackievirus A4 polyclonal antibody, an anti-coxsackievirus A5 polyclonal antibody, an anti-coxsackievirus A6 polyclonal antibody, and an anti-coxsackievirus A10 polyclonal antibody; a second reagent comprising the anti-coxsackievirus A2 polyclonal antibody; a third reagent comprising the anti-coxsackievirus A4 polyclonal antibody; a fourth reagent comprising the anti-coxsackievirus A5 polyclonal antibody; a fifth reagent comprising the anti-coxsackievirus A6 polyclonal antibody; a sixth reagent comprising the anti-coxsackievirus A10 polyclonal antibody; and a seventh reagent comprising a secondary antibody labeled with a fluorescence compound, wherein the secondary antibody is used for detecting the antibody anti-coxsackieviruses A2, A4, A5, A6 and A10 polyclonal antibodies and a titer of the anti-coxsackieviruses A2, A4, A5, A6 or A10 polyclonal antibody is about 1:5000-151:70000.

### BACKGROUND OF THE INVENTION

1. Field of the Invention

The present invention relates to a typing kit for virus, and in particular relates to an indirect immunofluorescence assay typing kit for coxsackievirus A group.

2. Description of the Related Art

The number of serotypes of enteroviruses is numerous (over 67) and traditional neutralization test for enterovirus typing is time consuming (about 5-7 days). Although molecular biological test may be used for determining the genotype of a virus, cost thereof is high. Therefore molecular biological tests are not popularly used in many clinical laboratories. In practice, the popular method for serotyping a viral isolate is indirect immunofluorescence assay (IFA). The advantages of IFA comprise convenience and faster results and the method may be used for typing large number of samples simultaneously.

However, commercially available IFA reagent provides limited coverage in enterovirus serotyping, currently only about 19 are available on the market, e.g., polio type 1, 2, and 3, coxsackievirus B1-B6, Echovirus 4, 6, 9, 11, 30, coxsackievirus A9, 16, 24, and enterovirus 70, 71.

Because there are differences in each country due to geography, latitude, weather, social customs and practices, economical background, and population distribution, etc., the epidemic trend and the prevalent serotype of enterovirus for each country are also different. Therefore a method which can be used to quickly and accurately type the serotype of enterovirus is urgently needed.

### BRIEF SUMMARY OF THE INVENTION

The present invention provides an indirect immunofluorescence assay typing kit for coxsackievirus, comprising: a first reagent comprising a mixture of an anti-coxsackievirus A2 polyclonal antibody, an anti-coxsackievirus A4 polyclonal antibody, an anti-coxsackievirus A5 polyclonal antibody, an anti-coxsackievirus A6 polyclonal antibody, and an anti-coxsackievirus A10 polyclonal antibody; a second reagent comprising the anti-coxsackievirus A2 polyclonal antibody; a third reagent comprising the anti-coxsackievirus A4 polyclonal antibody; a fourth reagent comprising the anti-coxsackievirus A5 polyclonal antibody; a fifth reagent comprising the anti-coxsackievirus A6 polyclonal antibody; a sixth reagent comprising the anti-coxsackievirus A10 polyclonal antibody; and a seventh reagent comprising a secondary antibody labeled with a fluorescence compound, wherein the secondary antibody is used for detecting the antibody anti-coxsackieviruses A2, A4, A5, A6 and A10 polyclone antibodies and a titer of the anti-coxsackieviruses A2, A4, A5, A6 or A10 polyclonal antibody is about 1:5000-1:70000.

The invention further provides a method for typing coxsackievirus A, comprising: (a) providing the indirect immunofluorescence assay typing kit mentioned above for a coxsackievirus; (b) providing a sample and observing the cytopathologic effect of the sample to determine that the sample is not infected with the herpes simplex virus (HSV); (c) treating the sample with the first reagent and then the seventh reagent to proceed with a first fluorescence stain reaction; (d) if the fluorescence stain reaction is positive, the sample is determined to be infected with the coxsackieviruses, A2, A4, A5, A6 or A10 and if the first fluorescence stain reaction is negative, the sample is determined to not be infected with the coxsackieviruses A2, A4, A5, A6 or A10; (e) after the step (d), treating the sample with the second, third, fourth, fifth and/or sixth reagent and then the seventh reagent, respectively, to proceed with a second fluorescence stain reaction; (f) if the second fluorescence stain reaction is positive after treating the sample with the second reagent and then the seventh reagent, the sample is determined to be infected with the coxsackievirus A2, and if the second fluorescence stain reaction is negative after treating the sample with the second reagent and then the seventh reagent, the sample is determined to be infected with one of the coxsackieviruses A4, A5, A6 and A10; (g) if the second fluorescence stain reaction is positive after treating the sample with the third reagent and then the seventh reagent, the sample is determined to be infected with the coxsackievirus A4, and if the second fluorescence stain reaction is negative after treating the sample with the third reagent and then the seventh reagent, the sample is determined to be infected with one of the coxsackieviruses A2, A5, A6 and A10; (h) if the second fluorescence stain reaction is positive after treating the sample with the fourth reagent and then the seventh reagent, the sample is infected with the coxsackievirus A5, and if the second fluorescence stain reaction is negative after treating the sample with the fourth reagent and then the seventh reagent, the sample is determined to be infected with one of the coxsackieviruses A2, A4, A6 and A10; (i) if the second fluorescence stain reaction is positive after treating the sample with the fifth reagent and then the seventh reagent, the sample is determined to be infected with the coxsackievirus A6, and if the second fluorescence stain reaction is negative after treating the sample with the fifth reagent and then the seventh reagent, the sample is determined to be infected with one of the coxsackieviruses A2, A4, A5 and A10; and (j) if the second fluorescence stain reaction is positive after treating the sample with the sixth reagent and then the seventh reagent, the sample is determined to be infected with the coxsackievirus A10, and if the second fluorescence stain reaction is negative after treating the sample with the sixth reagent and then the seventh reagent, the sample is determined to be infected with one of the coxsackieviruses A2, A4, A5 and A6.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a flow chart for using the indirect immunofluorescence assay typing kit for coxsackievirus A of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

First, coxsackieviruses A2, A4, A5, A6 and A10 identified by a neutralization test is chosen and proliferated, respectively. Then, the coxsackieviruses A2, A4, A5, A6 or A10 are used to immunize individual mammals of the same species, respectively, to obtain individual polyclonal antibodies. The mammals may comprise horses, monkeys, guinea pigs, mice or rabbits, with rabbits being more preferably. A titer of the anti-coxsackieviruses A2, A4, A5, A6 or A10 polyclonal antibody obtained through the method mentioned previously is about 1:5000-1:70000. The anti-coxsackieviruses A2, A4, A5, A6 or A10 polyclonal antibody are calibrated and prepared according to the checkerboard dilution test results, respectively, to form the second, third, fourth, fifth and sixth reagent, respectively. The appropriate concentration of each polyclonal antibody may be about 1:600-1-2000. The anti-coxsackieviruses-A2, A4, A5, A6 and A10 polyclonal antibodies are mixed to form a first reagent. Preferably, the anti-coxsackieviruses A2, A4, A5, A6 and A10 polyclonal antibodies are diluted by the ratio of 1:200, 1:150, 1:400, 1:200 and 1:200, respectively, and after dilution, the anti-coxsackieviruses A2, A4, A5, A6 and A10 polyclonal antibodies are mixed by the ratio of 1:1:1:1 to form the first reagent.

A seventh reagent comprising a secondary antibody labeled with a fluorescence compound, wherein the secondary antibody is used for detecting the antibody anti-coxsackieviruses A2, A4, A5, A6 and A10 polyclonal antibodies. The secondary antibody may be directed against an immunoglobuin of the mammal. According to the kind of mammal immunized to obtain the polyclonal antibodies, the secondary antibody may be directed against an immunoglobuin of horses, monkeys, guinea pigs, mice or rabbits. For example, if the polyclonal antibody is obtained from a rabbit, the secondary antibody is directed against an immunoglobuin of rabbits. The fluorescence compound of the secondary antibody may comprise fluoresceinisothiocynate (FITC), etc. The first to seventh reagents form the indirect immunofluorescence assay typing kit for coxsackievirus A of the invention.

The kit of the invention may be used for typing coxsackievirus A. Following, fluorescence stain reaction steps for samples will be described in more detail with reference to the Examples.

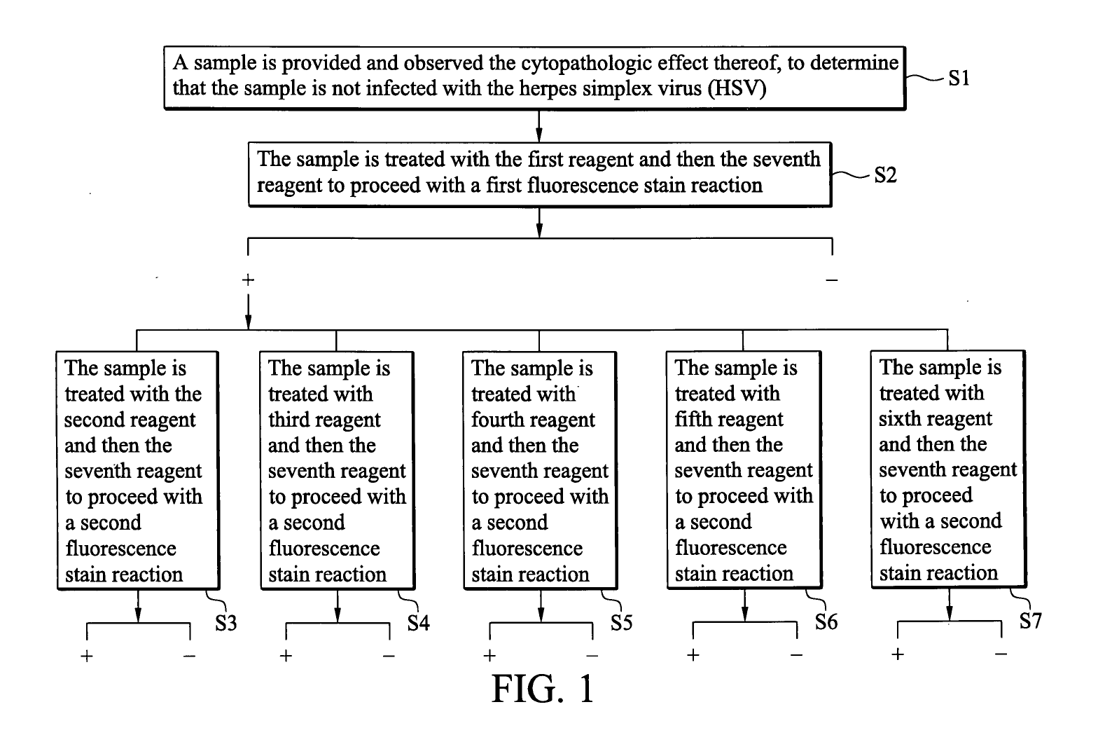

FIG. 1 shows a flow chart for using the indirect immunofluorescence assay typing kit for coxsackievirus A of the invention. First, a sample is provided and observed the cytopathologic effect thereof, to determine that the sample is not infected with the herpes simplex virus (HSV) (step S1). Note that for the first fluorescence stain reaction procedure for a herpes simplex virus (HSV), the result will be a false positive. However, the cytopathologic effect between the cell infected by the herpes simplex virus and enterovirus is different and the two viruses may be distinguished by the cytopathologic effect of the sample. Then the sample is treated with the first reagent and then the seventh reagent to proceed with a first fluorescence stain reaction (step S2). If the fluorescence stain reaction is positive, the sample is determined to be infected with the coxsackieviruses A2, A4, A5, A6 or A10 and if the first fluorescence stain reaction is negative, the sample is determined to not be infected with the coxsackieviruses A2, A4, A5, A6 or A10.

Next, a second fluorescence stain reaction procedure is conducted for positive ( +) fluorescence stain samples not infected by the herpes simplex virus. During the second fluorescence stain reaction procedure, the samples are treated with the second reagent and then the seventh reagent (step S3), third reagent and then the seventh reagent (step S4), fourth reagent and then the seventh reagent (step S5), fifth reagent and then the seventh reagent (step S6) and/or sixth reagent and then the seventh reagent (step S7). Note that the order of steps S3-S6 and step requirements are dependent upon requirements and steps S3-S6 may also be proceeded at the same time. Also, steps are required until the second fluorescence stain reaction is positive (+) and serotype of coxsackievirus A is identified.

If the second fluorescence stain reaction is positive (+) after the sample treated with the second reagent and then the seventh reagent, the sample is determined to be infected with the coxsackievirus A2. If the second fluorescence stain reaction is negative ( −) after the sample treated with the second reagent and then the seventh reagent, the sample is determined to be infected with one of the coxsackieviruses A4, A5, A6 and A10.

If the second fluorescence stain reaction is positive (+) after the sample treated with the third reagent and then the seventh reagent, the sample is determined to be infected with the coxsackievirus A4. If the second fluorescence stain reaction is negative (−) after the sample treated with the third reagent and then the seventh reagent, the sample is determined to be infected with one of the coxsackieviruses A2, A5, A6 and A10.

If the second fluorescence stain reaction is positive (+) after the sample treated with the fourth reagent and then the seventh reagent, the sample is determined to be infected with the coxsackievirus A5. If the second fluorescence stain reaction is negative ( −) after the sample was treated with the fourth reagent and then the seventh reagent, the sample is determined to be infected with one of the coxsackieviruses A2, A4, A6 and A10.

If the second fluorescence stain reaction is positive (+) after the sample treated with the fifth reagent and then the seventh reagent, the sample is determined to be infected with the coxsackievirus A6. If the second fluorescence stain reaction is negative (−) after the sample treated with the fifth reagent and then the seventh reagent, the sample is determined to be infected with one of the coxsackieviruses A2, A4, A5 and A10.

If the second fluorescence stain reaction is positive (+) after the sample treated with the sixth reagent and then the seventh reagent, the sample is determined to be infected with the coxsackievirus A10. If the second fluorescence stain reaction is negative (−) after the sample treated with the sixth reagent and then the seventh reagent, the sample is determined to be infected with one of the coxsackieviruses A2, A4, A5 and A6.

The samples mentioned previously may comprise a cell. In one embodiment, the secondary antibody of the seventh reagent is labeled with fluoresceinisothiocynate, wherein if the first or second fluorescence stain reaction is positive, a cell nucleus and a cytoplasm of the cell presents an apple green color, and if the first or second fluorescence stain reaction is negative the cell presents a red color.

### EXAMPLE

Preparation of the First to Seventh Reagents

Rabbits were used as a source to produce polyclonal antibodies. Coxsackieviruses A2, A4, A5, A6 and A10 identified by a neutralization test were chosen and proliferated, respectively. Then the viruses mentioned previously were treated with CHC1₃ and irradiation by UV light to inactivate. Four rabbits were grouped together and polyclonal antibody of each virus was prepared. The rabbits of every group were immunized with inactivated virus for 5 times, once every other day. Each dosage was 5 ml, and every ml contained 10⁸ CCID or larger. On the forty second day, the rabbits of every group were given 10 ml of non-deactivized viruses. After one week, blood was collected from the rabbits of every group to obtain anti-coxsackieviruses A2, A4, A5, A6 and A10 polyclonal antibodies, respectively.

Next, a neutralization test to determine the homotiter and heterotiter for different enteroviruses was performed on each polyclonal antibody. Note that the titer of anti-coxsackieviruses A2, A4, A5, A6 or A10 polyclonal antibody was about 1:5000-1:70000.

Following, the appropriate concentration of anti-coxsackieviruses A2, A4, A5, A6 and A10, polyclonal antibodies were calibrated and prepared according to the checkerboard dilution test results, respectively. As a result, the polyclonal antibodies formed the second, third, fourth, fifth and sixth reagents, respectively and the appropriate concentrations of anti-coxsackieviruses A2, A4, A5, A6 and A10 polyclonal antibodies were 1:1000, 1:600, 1:2000, 1:1000 and 1:1000, respectively.

Anti-coxsackieviruses A2, A4, A5, A6 and A10 polyclonal antibodies were diluted by the ratio of 1:200, 1:150, 1:400, 1:200 and 1:200, respectively and after dilution, the anti-coxsackieviruses A2, A4, A5, A6 and A10 polyclonal antibodies were mixed by the ratio of 1:1:1:1:1 to form the first reagent.

Next, anti-rabbit immunoglobulin G was provided and labeled with fluoresceinisothiocynate as a secondary antibody or commercially available anti-rabbit immunoglobulin G labeled with fluoresceinisothiocynate was obtained as a secondary antibody. The appropriate concentration of anti-rabbit immunoglobulin G labeled with fluoresceinisothiocynate was determined to form the seventh reagent.

Performance Test

The calculation method for sensitivity and specificity of the first through the sixth reagents is shown in Table 1.

TABLE 1
The calculation method for sensitivity and specificity of the first
through the sixth reagents

| | The sample has been | The sample has been |
|---|---|---|
| | identified to be infected | identified to be infected with |
| | with the serotype of virus | the serotype of virus which |
| | which matches the reagent | does not matches the reagent |
| Fluorescence | a (number of samples) | b (number of samples) |
| stain reaction | | |
| (positive) | | |
| Fluorescence | c (number of samples) | d (number of samples) |
| stain reaction | | |
| (negative) | | |

* Sensitivity = a/(a+c)
* Specificity = d/(b+d)

The data for evaluation of sensitivity and specificity of the first through the sixth reagents was obtained by testing the clinical isolation strains of the enteroviruses prevalent in Taiwan in 1998-2006. The data of the first through the sixth reagents is shown in Tables 2-7, respectively.

TABLE 2
The samples treated with the first reagent and then the seventh reagent
which were proceeded with fluorescence stain reactions

| | The sample has been | The sample has been |
|---|---|---|
| | identified to be infected with | identified to be not infected |
| | the coxsackievirus A2, 4, 5, | with the coxsackievirus A2, |
| | 6 or 10 | 4, 5, 6 or 10 |
| Fluorescence | 100 (a) | 6 (b) |
| stain reaction | | |
| (positive) | | |
| Fluorescence | 0 (c) | 128 (d) |
| stain reaction | | |
| (negative) | | |

TABLE 3
The samples treated with the second reagent and then the seventh
reagent which were proceeded with fluorescence stain reactions

| | The sample has been | The sample has been |
|---|---|---|
| | identified to be infected with | identified to be not infected |
| | the coxsackievirus A2 | with the coxsackievirus A2 |
| Fluorescence | 87 (a) | 7 (b) |
| stain reaction | | |
| (positive) | | |
| Fluorescence | 0 (c) | 172 (d) |
| stain reaction | | |
| (negative) | | |

TABLE 4
The samples treated with the third reagent and then the seventh reagent
which were proceeded with fluorescence stain reactions

| | The sample has been | The sample has been |
|---|---|---|
| | identified to be infected with | identified to be not infected |
| | the coxsackievirus A4 | with the coxsackievirus A4 |
| Fluorescence | 209 (a) | 7 (b) |
| stain reaction | | |
| (positive) | | |
| Fluorescence | 9 (c) | 206 (d) |
| stain reaction | | |
| (negative) | | |

TABLE 5
The samples treated with the fourth reagent and then the seventh
reagent which were proceeded with fluorescence stain reactions

| | The sample has been | The sample has been |
|---|---|---|
| | identified to be infected with | identified to be not infected |
| | the coxsackievirus A5 | with the coxsackievirus A5 |
| Fluorescence | 46 (a) | 7 (b) |
| stain reaction | | |
| (positive) | | |
| Fluorescence | 0 (c) | 158 (d) |
| stain reaction | | |
| (negative) | | |

TABLE 6
The samples treated with the fifth reagent and then the seventh reagent
which were proceeded with fluorescence stain reactions

| | The sample has been | The sample has been |
|---|---|---|
| | identified to be infected with | identified to be not infected |
| | the coxsackievirus A6 | with the coxsackievirus A6 |
| Fluorescence | 73 (a) | 4 (b) |
| stain reaction | | |
| (positive) | | |
| Fluorescence | 0 (c) | 140 (d) |
| stain reaction | | |
| (negative) | | |

TABLE 7
The samples treated with the sixth reagent and then the seventh reagent
which were proceeded with fluorescence stain reactions

| | | The sample |
|---|---|---|
| | The sample has been | has been identified |
| | identified to be infected with | to be infected with |
| | the coxsackievirus A10 | the coxsackievirus A10 |
| Fluorescence | 96 (a) | 6 (b) |
| stain reaction | | |
| (positive) | | |
| Fluorescence | 3 (c) | 147 (d) |
| stain reaction | | |
| (negative) | | |

The sensitivity and specificity of the first through the sixth reagents were calculated based upon the data shown in Tables 2-7 by the method shown in Table 1, and the results thereof are shown in Table 8.

TABLE 8
The sensitivity and specificity of the first through the sixth reagents

| | A2, A4, | | | | | |
|---|---|---|---|---|---|---|
| | A5, A6 | | | | | |
| | and A10 | A2 | A4 | A5 | A6 | |
| | (first | (second | (third | (fourth | (fifth | A10 (sixth |
| Serotype | reagent) | reagent) | reagent) | reagent) | reagent) | reagent) |
| Sensitivity | 100 | 100 | 95.59 | 100 | 100 | 96.59 |
| (%) | | | | | | |
| Specificity | 95.5 | 96.1 | 96.9 | 95.8 | 96.6 | 96.1 |
| (%) | | | | | | |

The Steps of Fluorescence Stain Reaction

Samples infected by an enterovirus were treated with the kit obtained through the method mentioned previously to proceed with fluorescence stain reactions.

The steps of fluorescence stain reaction comprise: (1) warming slides with samples in wells to reach room temperature; (2) adding the first reagent in every well (the first reagent has to cover the whole well and according with the diameter of the well, 8-10 μl of the first reagent is added to the well); (3) incubating slides with samples at 37° C. for 30 minutes; (4) washing the slides with PBST (pH 7.2-7.4, containing 0.05% Tween ) and drying the slides at room temperature; (5) adding the seventh reagent in every well (the seventh reagent has to cover the whole well and according with the diameter of the well, 8-10 μl of the seventh reagent is added to the well); (6) incubating slides with samples at 37° C. for 30 minutes; (7) washing the slides with PBST; (8) adding appropriate amounts of mounting oil and covering the cover glass on the slide; and (9) observing the samples by fluorescence microscopy: if fluorescence stain reaction is positive, a cell nucleus and a cytoplasm of the cell of the sample presents an apple green color, and if the fluorescence stain reaction is negative the cell of the sample presents a red color. Note that for samples with positive (+) fluorescence stain reaction, steps (2)-(9) must be repeated using new and identical samples, wherein second to sixth reagents respectively replace the first reagent.

The Fluorescence Stain Reaction for Different Samples

Coxsackieviruses A2, A4, A5, A6 and A10 were incoculated onto RD cells, respectively, to form 5 samples. When the cells presented cytopathologic effect, the samples were treated with the first reagent and then seventh reagent to proceed with fluorescence stain reactions (the first fluorescence stain). The result showed that the fluorescence stain reactions of five samples were all positive and presented an apple green color. As such, the first reagent of the kit had high sensitivity for coxsackieviruses A2, A4, A5, A6 and A10.

Coxsackieviruses A2, A4, A5, A6 and A10 were infected on RD cells, respectively, to form five samples. When the cells presented cytopathologic effect, the samples were treated with the second to sixth reagents, respectively and then seventh reagent to proceed with fluorescence stain reactions (the second fluorescence stain). The results showed that the fluorescence stain reactions of five samples were all positive and presented an apple green color. As such, the second to sixth reagents of the kit had high sensitivity for coxsackieviruses A2, A4, A5, A6 and A10, respectively.

Additionally, coxsackievirus A16 and enterovirus 71 were infected in RD cells, respectively, to form two samples. The samples were treated with the first reagent, and then seventh reagent to proceed with fluorescence stain reactions (the first fluorescence stain). The results showed that the fluorescence stain reactions of the two samples were both negative and presented a red color. As such, the first reagent of the kit has high specificity.

Furthermore, RD cell, Hep-2c cell and MK2 cells which were not infected with the virus, were respectively treated with the first reagent, and then seventh reagent to proceed with fluorescence stain reactions (the first fluorescence stain). The results showed that the fluorescence stain reactions of the three kinds of cells were all negative and presented a red color. As such, the first reagent of the kit had high specificity.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An indirect immunofluorescence assay typing kit for coxsackievirus, comprising:
a first reagent comprising a mixture of an anti-coxsackievirus A2 polyclonal antibody, an anti-coxsackievirus A4 polyclonal antibody, an anti-coxsackievirus A5 polyclonal antibody, an anti-coxsackievirus A6 polyclonal antibody, and an anti-coxsackievirus A10 polyclonal antibody;
a second reagent comprising the anti-coxsackievirus A2 polyclonal antibody;
a third reagent comprising the anti-coxsackievirus A4 polyclonal antibody;
a fourth reagent comprising the anti-coxsackievirus A5 polyclonal antibody;
a fifth reagent comprising the anti-coxsackievirus A6 polyclonal antibody;
a sixth reagent comprising the anti-coxsackievirus A10 polyclonal antibody; and
a seventh reagent comprising a secondary antibody labeled with a fluorescence compound,
wherein the secondary antibody is used for detecting the antibody anti-coxsackieviruses A2, A4, A5, A6 and A10 polyclonal antibodies and a titer of the anti-coxsackieviruses A2, A4, A5, A6 or A10 polyclonal antibody is about 1:5000-1:70000.

2. The indirect immunofluorescence assay typing kit for coxsackievirus as claimed in claim 1, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a mammal, and the secondary antibody is directed against an immunoglobuin of the mammal.

3. The indirect immunofluorescence assay typing kit for coxsackievirus as claimed in claim 1, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a horse, a monkey, a guinea pig, a mouse or a rabbit, and the secondary antibody is directed against an immunoglobuin of the horse, the monkey, the guinea pig, the mouse or the rabbit.

4. The indirect immunofluorescence assay typing kit for coxsackievirus as claimed in claim 1, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a rabbit, and the secondary antibody is directed against an immunoglobuin of the rabbit.

5. The indirect immunofluorescence assay typing kit for coxsackievirus as claimed in claim 1, wherein the fluorescence compound comprising fluoresceinisothiocynate.

6. The invention further provides a method for typing coxsackievirus A, comprising:
(a) providing the indirect immunofluorescence assay typing kit as claimed in claim 1 for a coxsackievirus;
(b) providing a sample and observing the cytopathologic effect of the sample to determine that the sample is not infected with the herpes simplex virus (HSV);
(c) treating the sample with the first reagent and then the seventh reagent to proceed with a first fluorescence stain reaction;
(d) if the fluorescence stain reaction is positive, the sample is determined to be infected with the coxsackieviruses A2, A4, A5, A6 or A10 and if the first fluorescence stain reaction is negative, the sample is determined to not be infected with the coxsackieviruses A2, A4, A5, A6 or A10;
(e) after the step (d), treating the sample with the second, third, fourth, fifth and/or sixth reagent and then the seventh reagent, respectively, to proceed with a second fluorescence stain reaction;
(f) if the second fluorescence stain reaction is positive after treating the sample with the second reagent and then the seventh reagent, the sample is determined to be infected with the coxsackievirus A2, and if the second fluorescence stain reaction is negative after treating the sample with the second reagent and then the seventh reagent, the sample is determined to be infected with one of the coxsackieviruses A4, A5, A6 and A10;
(g) if the second fluorescence stain reaction is positive after treating the sample with the third reagent and then the seventh reagent, the sample is determined to be infected with the coxsackievirus A4, and if the second fluorescence stain reaction is negative after treating the sample with the third reagent and then the seventh reagent, the sample is determined to be infected with one of the coxsackieviruses A2, A5, A6 and A10;
(h) if the second fluorescence stain reaction is positive after treating the sample with the fourth reagent and then the seventh reagent, the sample is infected with the coxsackievirus A5, and if the second fluorescence stain reaction is negative after treating the sample with the fourth reagent and then the seventh reagent, the sample is determined to be infected with one of the coxsackieviruses A2, A4, A6 and A10;
(i) if the second fluorescence stain reaction is positive after treating the sample with the fifth reagent and then the seventh reagent, the sample is determined to be infected with the coxsackievirus A6, and if the second fluorescence stain reaction is negative after treating the sample with the fifth reagent and then the seventh reagent, the sample is determined to be infected with one of the coxsackieviruses A2, A4, A5 and A10; and
(j) if the second fluorescence stain reaction is positive after treating the sample with the sixth reagent and then the seventh reagent, the sample is determined to be infected with the -coxsackievirus A10, and if the second fluorescence stain reaction is negative after treating the sample with the sixth reagent and then the seventh reagent, the sample is determined to be infected with one of the coxsackieviruses A2, A4, A5 and A6.

7. The method for typing coxsackievirus A as claimed in claim 6, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a mammal, and the secondary antibody is directed against an immunoglobuin of the mammal.

8. The method for typing coxsackievirus A as claimed in claim 6, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a horse, a monkey, a guinea pig, a mouse or a rabbit, and the secondary antibody is directed against an immunoglobulin of the horse, the monkey, the guinea pig, the mouse or the rabbit.

9. The method for typing coxsackievirus A as claimed in claim 6, wherein the anti-coxsackievirus A2 polyclonal antibody, anti-coxsackievirus A4 polyclonal antibody, anti-coxsackievirus A5, polyclonal antibody, anti-coxsackievirus A6 polyclonal antibody and anti-coxsackievirus A10 polyclonal antibody are obtained by immunizing a rabbit, and the secondary antibody is directed against an immunoglobulin of the rabbit.

10. The method for typing coxsackievirus A as claimed in claim 6, wherein the sample comprises a cell.

11. The method for typing coxsackievirus A as claimed in claim 10, wherein the fluorescence compound comprises fluoresceinisothiocynate.

12. The method for typing coxsackievirus A as claimed in claim 11, wherein if the first or second fluorescence stain reaction is positive, a cell nucleus and a cytoplasm of the cell presents an apple green color, and if the first or second fluorescence stain reaction is negative the cell presents a red color.
